(19) United States
(12) Patent Application Publication
(10) Pub. No.: US 2010/0286101 A1
(43) Pub. Date: Nov. 11, 2010

(54) PHARMACEUTICAL COMPOSITION INCLUDING A CORTICOSTEROID AND A VITAMIN D ANALOG HAVING IMPROVED STABILITY

(76) Inventors: Jason Carbol, Fort Collins, CO (US); Herbert Brinkman, Fort Collins, CO (US)

Correspondence Address: SCHWEGMAN, LUNDBERG & WOESSNER, P.A., P.O. BOX 2938, MINNEAPOLIS, MN, 55402, US

(21) Appl. No.: 12/776,111

(22) Filed: May 7, 2010

Related U.S. Application Data
(60) Provisional application No. 61/325,857, filed on Apr. 20, 2010.
(60) Provisional application No. 61/176,269, filed on May 7, 2009.

(51) Int. Cl. A61K 31/593; A61P 17/00

(52) U.S. Cl. 514/167

(57) ABSTRACT

A composition that includes a corticosteroid (e.g., betamethasone dipropionate), a vitamin D analog (e.g., calcipotriene) and an acyl ester of 1,2,3-trihydroxypropane or an ether thereof; and a method of treating a dermatologic condition (e.g., psoriasis vulgaris) in a mammal (e.g., human); are provided. The method includes topically administering to a mammal in need of such treatment an effective amount of the composition, to the affected topical area, for a period of time effective to treat the dermatologic condition.

### Cross-Reference to Related Applications

This application claims the priority of U.S. Provisional Application Ser. Number 61/176,269, filed May 7, 2009, and of U.S. Provisional Application Ser. No. 61/325,857, filed Apr. 20, 2010, which are incorporated herein by reference in their entirety.

### BACKGROUND OF THE INVENTION

Psoriasis and other dermatologic conditions are often treated by topically applying a corticosteroid in combination with vitamin D or an analog thereof. See, for example, Didriksen, U.S. Pat. No. 6,753,013, (incorporated herein by reference in its entirety) which describes certain formulations incorporating both the corticosteroid and the vitamin D analog.

As stated in Didriksen, due to chemical instability of this combination in certain formulations, physicians were forced to resort to letting patients who were being treated with a regimen of a corticosteroid and vitamin D or an analog thereof perform sequential application of two compositions, one containing the corticosteroid and the other containing the vitamin D or analog. Under such circumstances, problems with patient compliance and correct administration of dosage were experienced. Didriksen provided certain water-in-oil or oil-in-water emulsion pharmaceutical compositions containing a corticosteroid and vitamin D or an analog thereof in which both of these components were stable.

### SUMMARY OF THE INVENTION

It has unexpectedly been discovered that chemically stable compositions including both a corticosteroid and vitamin D analog are obtained by dispersing the bioactive components in various embodiments of a solvent system of the invention. In various embodiments, the invention provides a composition comprising:

(a) a corticosteroid;

(b) a vitamin D analog; and

(c) an acylated glycerol.

The solvent system can include one or more specific acylated glycerols as defined herein, each of which independently can possess a relatively low dipole, a relatively large degree of hydrogen bonding, and relatively high degree of oil miscibility (e.g., not miscible in water). A solvent system of the invention can be substantially free of water. Each of the corticosteroid and vitamin D analog can be suspended, dissolved, dispersed, or emulsified in the inventive composition, which has unexpected been found to confer enhanced stability on the active pharmaceutical ingredients of the composition.

The corticosteroid and vitamin D analog are chemically stable in the composition, e.g., when stored at 40° C. for a period of 4 weeks, or in various embodiments at 50° C. for a similar period.

The composition described herein can be suitable for topical administration. In various embodiments, the composition can be, e.g., a cream, gel, lotion or ointment. Alternatively, the composition can be present on a topical skin patch.

The present invention also provides for a method of treating a dermatologic condition in a mammal. The method includes topically administering to a mammal in need of such treatment an effective amount of the composition described herein, to the affected topical area, for a period of time effective to treat the dermatologic condition. The dermatologic condition can be psoriasis vulgaris.

The administration can be one to about three times daily. Additionally, the administration can be for up to about four weeks.

In a specific embodiment, the maximum weekly dose does not exceed about 100 grams. In another specific embodiment, the composition is administered to no more than about 30% of the body surface area of the mammal.

The mammal can be an adult, 18 years of age or older.

The present invention also provides for a method for making a composition that includes a corticosteroid, a vitamin D analog and an acylated glycerol as the term is defined herein. The corticosteroid and the vitamin D analog can be suspended, dissolved, dispersed, or emulsified in the acylated glycerol. In various embodiments, water can be substantially excluded from the inventive composition.

### DETAILED DESCRIPTION OF THE INVENTION

Reference will now be made in detail to certain claims of the invention, examples of which are illustrated in the accompanying structures and formulas. While the invention will be described in conjunction with the enumerated claims, it will be understood that they are not intended to limit the invention to those claims. On the contrary, the invention is intended to cover all alternatives, modifications, and equivalents, which may be included within the scope of the invention as defined by the claims.

References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The invention relates to a composition that includes a corticosteroid, a vitamin D analog and a solvent comprising an acylated glycerol within the meaning of the term as defined herein; and to a method of treating a dermatologic condition in a mammal that includes topically administering the composition. When describing the composition and use of the composition, the following terms have the following meanings, unless otherwise indicated.

### Definitions

Unless stated otherwise, the following terms and phrases as used herein are intended to have the following meanings:

When trade names are used herein, applicants intend to independently include the trade name product and the active pharmaceutical ingredient(s) of the trade name product.

As used herein, the term “corticosteroid” refers to a class of steroid hormones that are produced in the adrenal cortex. Corticosteroids are involved in a wide range of physiologic systems such as stress response, immune response and regulation of inflammation, carbohydrate metabolism, protein catabolism, blood electrolyte levels, and behavior.

Corticosteroids are generally grouped into four classes, based on chemical structure. Group A corticosteroids (short to medium acting glucocorticoids) include hydrocortisone, hydrocortisone acetate, cortisone acetate, tixocortol pivalate, prednisolone, methylprednisolone, and prednisone. Group B corticosteroids include triamcinolone acetonide, triamcinolone alcohol, mometasone, amcinonide, budesonide, desonide, fluocinonide, fluocinolone acetonide, and halcinonide. Group C corticosteroids include betamethasone, betamethasone sodium phosphate, dexamethasone, dexamethasone sodium phosphate, and fluocortolone. Group D corticosteroids include hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate.

Corticosteroids include topical steroids, which have anti-inflammatory properties, and are classified based on their vasoconstriction abilities. In the United States, the topical steroids are grouped into seven classes in which Class I is the strongest and Class VII is the weakest. Topical steroid group I includes clobetasol diproprionate 0.05%, betamethasone diproprionate 0.25%, halbetasol proprionate 0.05% and diflorasone diacetate 0.05%. Topical steroid group II includes fluocinonide 0.05%, halcinonide 0.05%, amcinonide 0.05% and desoximetasone 0.25%. Topical steroid group III includes triamcinolone acetonide 0.5%, mometasone furoate 0.1%, fluticasone proprionate 0.005% and betamethasone diproprionate 0.05%. Topical steroid group IV includes fluocinolone acetonide 0.01-0.2%, hydrocortisone valerate 0.2%, hydrocortisone butyrate 0.1%, flurandrenolide 0.05%, triamcinolone acetonide 0.1% and mometasone furoate 0.1%. Topical steroid group V includes triamcinolone acetonide 0.1%, fluticasone propionate 0.05%, desonide 0.05%, fluocinolone acetonide 0.025% and hydrocortisone valerate 0.2%. Topical steroid group VI includes prednicarbate 0.05%, triamcinolone acetonide 0.025%, fluocinolone acetonide 0.01% and desonide 0.05%. Topical steroid group VII includes hydrocortisone 2.5% and hydrocortisone 1%.

Suitable exemplary corticosteroids include betamethasone, betamethasone-21-acetate, betamethasone-17-adamantoate, betamethasone-17-benzoate, betamethasone-17-valerate, betamethasone-17,21-dipropionate, alclomethasone, alclomethasone dipropionate, clobetasol, clobetasol propionate, clobetasone, clobetasone-17-butyrate, desoximetasone, diflucortolone, diflucortolone-21-valerate, diflorasone, diflorasone diacetate, fluocinonide, flumetasone, flumetasone pivalate, fluocinolone, fluocinolone acetonide, fluticasone, fluticasone propionate, fluprednidene, fluprednidene acetate, halcinonide, hydrocortisone, hydrocortisone-17-butyrate, mometasone, mometasone furoate, triamcinolone, triamcinolone acetonide (acetonide-21-N-benzoyl-2-methyl-β-alaninate) or triamcinolone acetonide (acetonide-21-(3,3-dimethylbutyrate). In a specific embodiment, the corticosteroid is betamethasone-17,21-dipropionate.

Additional suitable exemplary corticosteroids are disclosed, e.g., in Goodman Gilman, Alfred; Goodman, Louis S.; Gilman, Alfred; Goodman and Gilman's The Pharmacological Basis of Therapeutics, Sixth Edition, pp.1482-1486; Christophers, Enno; Schöpf, Erwin; Kligman, Albert M.; Stoughton, Richard B.; and Topical Corticosteroid Therapy; A Novel Approach to Safer Drugs, Raven Press, pp. 3-5.

Additional suitable exemplary corticosteroids are disclosed in U.S. Pat. No. 6,579,512.

The term “corticosteroids” includes all suitable hydrates, pharmaceutically acceptable salts and polymorphs thereof.

“Betamethasone dipropionate” is a synthetic corticosteroid having the chemical name 9-fluoro-11(β),17,21-trihydroxy-16(β)-methylpregna-1,4-diene-3,20-dione17,21-dipropionate, with the empirical formula C₂₈H₃₇FO₇, a molecular weight of 504.59, and the following structural formula:

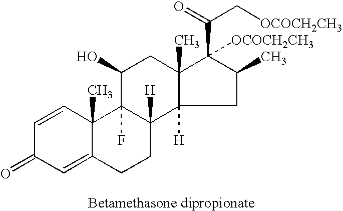

The term “betamethasone dipropionate” includes all suitable hydrates, pharmaceutically acceptable salts and polymorphs thereof.

As used herein, the term “vitamin D analog” refers to a group of fat-soluble prohormones, the two major forms of which are vitamin D₂ (or ergocalciferol) and vitamin D₃ (or cholecalciferol). The term vitamin D also refers to metabolites and other analogs of these substances. Vitamin D₃ is produced in skin exposed to sunlight, specifically ultraviolet B radiation. Several forms (vitamers) of vitamin D have been discovered (e.g., Vitamin D₁ or a molecular compound of ergocalciferol with lumisterol, 1:1; Vitamin D₂ or ergocalciferol; Vitamin D₃ or cholecalciferol; Vitamin D₄ or 22-dihydroergocalciferol; and Vitamin D₅ or sitocalciferol). The two major forms are vitamin D₂ or ergocalciferol, and vitamin D₃ or cholecalciferol. These are known collectively as calciferol. Chemically, the various forms of vitamin D are secosteroids; e.g., steroids in which one of the bonds in the steroid rings is broken. Additional suitable exemplary vitamin D analogs are disclosed in U.S. Pat. No. 6,753,013.

The term “vitamin D analog” includes all suitable hydrates, pharmaceutically acceptable salts and polymorphs thereof.

“Calcipotriene” or “calcipotriol” is a Vitamin D₃ analog having the chemical name (5Z,7E,22E,24S)-24-cyclopropyl-9,10-secochola-5,7,10(19), 22-tetraene-1α,3β,24-triol, with the empirical formula C₂₇H₄₀O₃, a molecular weight of 412.60, and the following structural formula:

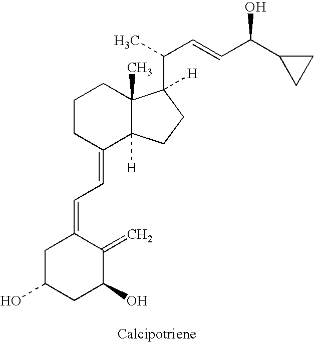

The term “calcipotriene” includes all suitable hydrates, pharmaceutically acceptable salts and polymorphs thereof.

As used herein, a “glycerol” refers to 1,2,3-trihydroxypropane or an ether thereof, wherein at least one free hydroxyl group is available for acylation, as described below. An ether of 1,2,3-trihydroxypropane can be a mono- or a di-alkyl ether, or a mono-, bis-, or tris-hydroxylalkyl ether, or any combination thereof. Accordingly, a glycerol within the meaning herein includes 1,2,3-trihydroxypropane, commonly known as glycerin:

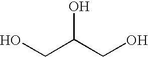

as is well known in the art.

A “glycerol” within the meaning herein also includes compounds of the structure

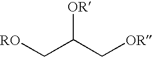

wherein each of R, R′, and R″ independently is selected from hydrogen, an alkyl group, or a hydroxyalkyl group, provided that the structure contains at least one free hydroxyl group available for acylation, e.g., formation of an ester bond with a carboxylic acid, as described below. Thus, if two of R, R′, and R″ are alkyl, then the third of these must be hydrogen or hydroxyalkyl, such that at least one free hydroxyl group of the glycerol is available for acylation. However, all of R, R′, and R″ can be hydrogen, and all can be hydroxyalkyl.

Accordingly, the methyl ether of 1,2,3-trihydroxypropane, e.g., 1-methoxy-2,3-dihydroxypropane

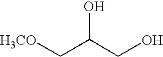

is a glycerol within the meaning herein. A dimethyl ether of 1,2,3-trihydroxypropane is also a glycerol within the meaning herein. However, a trimethyl ether of 1,2,3-trihydroxypropane, e.g., 1,2,3-trimethoxypropane, is excluded, as it does not contain at least one free hydroxyl group available for formation of an ester bond with an acyl group.

Similarly, a higher alkyl ether such as an ethyl ether, or an n-propyl ether, of 1,2,3-trihydroxypropane, is a glycerol within the meaning herein. A hydroxyalkyl ether of 1,2,3-trihydroxypropane is a glycerol within the meaning herein. For example, a mono-hydroxyethyl ether of 1,2,3-trihydroxypropane

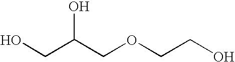

is a glycerol within the meaning herein. Or, a mono-hydroxypropyl ether of 1,2,3-trihydroxypropane

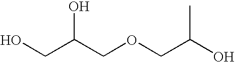

is a glycerol within the meaning herein.

A bis-hydroxyalkyl ether,

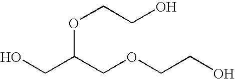

and a tris-hydroxyalkyl ether,

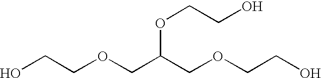

of 1,2,3-trihydroxypropane are examples of a glycerol within the meaning herein. A trishydroxyalkyl ether of 1,2,3-trihydroxypropane does contain at least one free hydroxyl group available for formation of an ester bond with an acyl group.

A hydroxyalkyl ether includes an oligomeric or a polymeric ether of 1,2,3-trihydroxypropane, such as a diethyleneglycol ether of 1,2,3-trihydroxypropane

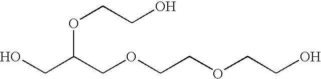

or a triethyleneglycol ether of 1,2,3-trihydroxypropane. In the same manner one or more of the three hydroxyl groups of 1,2,3-trihydroxypropane can be substituted with hydroxyalkyl groups including oligomeric or polymeric forms, such as a PEGylated (mono-, di-, or tri substituted) 1,2,3-trihydroxypropane. A PEGylated compound, as is well known in the art, is a compound to which a polyethyleneglycol (polyoxyethylene) chain has been covalently bonded

An “acyl” group refers to an organic moiety bearing a carbonyl (C═O) group, e.g., a group or moiety of the structure R¹C(═O). An acylated group is thus a group to which an acyl group is bound, e.g. a group bound to a carbonyl group, which carbonyl group the organic moiety R¹ is bonded. For example, a hydroxyl group of an alcohol such as R²—OH may be acylated, thus forming an ester bond.

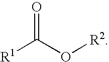

In these structural representations, R¹ and R² each independently signifies an organic moiety such as alkyl group, an aryl group, an aralkyl group, and the like; and wherein the R¹C(═O) group is an acyl group and wherein the R²—O group is an alkoxyl group.

Examples of an acyl group, shown as the R¹(═O) group above include an acetyl group (CH₃C(═O)) group wherein R¹ is methyl, a hexanoyl group (CH₃(CH₂)₄C(═O)) also known as a caproic group wherein R¹ is pentyl, an octanoyl group (CH₃(CH₂)₆C(═O)) also known as a caprylic group wherein R¹ is heptyl, a decanoyl group (CH₃(CH₂)₈C(=O)) also known as a capric group, wherein R¹ is nonyl, and so forth. An acyl group can also be a benzoyl group, an alkylbenzoyl group such as a toluoyl group, and the like, as is well known in the art.

Therefore, an “acylated glycerol” as the term is used herein refers to a glycerol within the meaning herein bonded via one or more ester bonds with one or more acyl groups as defined herein at one or more free hydroxyl groups. For example, a mono-acetylglycerol, a diacetylglycerol, or a triacetylglycerol is each an acylated glycerol within the meaning herein. Because a glycerol within the meaning herein includes an etherified 1,2,3-trihydroxypropane, such as a hydroxyethylated 1,2,3-trihydroxypropane, an acylated glycerol also includes acylated forms of hydroxyethylated 1,2,3-trihydroxypropane, wherein the acyl group(s) can be bonded to a hydroxyl group of the 1,2,3-trihydroxypropane fragment or a hydroxyl group of the hydroxyalkyl (e.g., hydroxyethyl) ether fragment, or both.

Some exemplary structures of acylated glycerols within the meaning herein are shown below.

An acylated glycerol of the formula

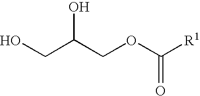

is an example of a 1-acylglycerol.

An acylated glycerol of the formula

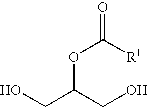

is an example of a 2-acylglycerol.

An acylated glycerol of the formula

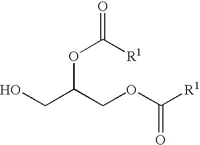

is an example of a 1,2-diacylglycerol.

An acylated glycerol of the formula

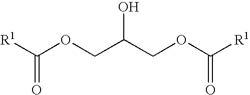

is an example of a 1,3-diacylglycerol.

An acylated glycerol of the formula

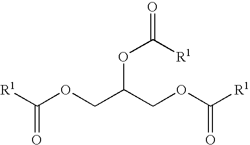

is an example of a 1,2,3-triacylglycerol, termed a “triglyceride.”

Etherified forms of 1,2,3-trihydroxypropane, such as hydroxyethylated 1,2,3-trihydroxypropane, are glycerols within the meaning herein, so acylated forms of these etherified compounds are also acylated glycerols within the meaning herein. Some exemplary acylated glycerols composed of etherified forms of 1,2,3-trihydroxypropane include the following.

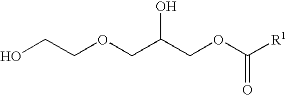

1-acyl-3-hydroxyethyl-1,2,3-trihydroxypropane;

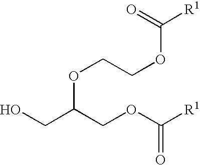

1-acyl-2-acyloxyethyl-1,2,3-trihydroxypropane;

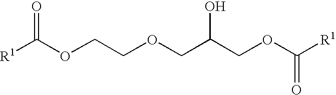

1-acyl-3-acyloxyethyl-1,2,3-trihydroxypropane;

wherein each R¹ can be independently selected from an organic group such as an alkyl, aryl, aralkyl, or other organic group.

Acylated glycerols include PEGylated acylated glycerols. A PEGylated example of an acylated glycerol within the meaning herein is shown as the following structure

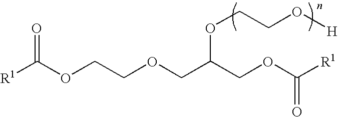

1-acyl-2-PEGyl-3-acyloxyethyl-1,2,3-trihydroxypropane.

wherein n indicates the degree of polymerization of the PEG (polyethyleneglycol) moiety, which can range from 2 up to at least about 100. In various embodiments, the terminal hydroxyl group of the PEG chain can also bear an acyl group.

Other permutations of acylated glycerols falling within the above definitions will occur to the person of ordinary skill in the art.

The solvents of the invention include such acylated glycerols and others apparent to those of skill in the art in which one or more hydroxyl groups of the glycerol, or one or more hydroxyl group of a hydroxyalkyl ether group, or both, bears an acyl group.

When one or more R¹ groups of the structures shown above are alkyl groups, the acyl groups that include the alkyl groups are termed “alkanoyl” groups, and the corresponding acylated glycerol is termed an “alkanoylated glycerol.” An “alkanoyl group” according to the meaning herein refers to an alkyl group bearing a carbonyl which can form an ester with a glycerol hydroxyl group. An alkyl group can be straight chain, branched, can include unsaturations such as double bonds, can include cycloalkyl groups, can include heteroatoms such as O, S, S(O), S(O)₂, or NR (wherein R can be H or simple alkyl) in the alkyl moiety. The alkyl group of an alkanoyl moiety can also include additional carbonyl groups in the alkyl chain, for example in the form of an amide group —C(O)NR— within the alkyl chain.

A “PEGylated” polyol is a polyol being a polyoxyalkylene group, such as a polyoxyethylene (polyethylene glycol, PEG) group as shown above

As used herein, the term “fatty acyl glycerol” refers to a glycerol, e.g., 1,2,3-trihydroxypropane or an ether thereof in which at least one hydroxyl group of the glycerol as defined herein is esterified with a fatty acyl group.

As used herein, the term “fatty acyl” refers to an acyl group derived from an liner aliphatic monocarboxylic acid containing about four to about 28 carbon atoms. The alkyl group of the fatty acyl group be saturated or unsaturated, but is unbranched.

As used herein, the term “monoglyceride” refers to esters formed from glycerol and a fatty acyl group in which only one hydroxyl group is esterified, such as in the examples of the 1-acylglycerol and 2-acylglycerol structures shown above.

As used herein, the term “diglyceride” refers to esters formed from glycerol and two independently selected fatty acyl groups in which two hydroxyl groups are esterified, such as for example in the case of the 1,2-diacylglycerol and 1,3-diacylglycerol structures shown above.

As used herein, the term “triglyceride” refers to esters formed from glycerol and three independently selected fatty acyl groups in which three hydroxyl groups are esterified.

As used herein, the term “a-monoglyceride” refers to an ester composed of a glycerol and an acyl group in which a hydroxyl group disposed on the 1-position of 1,2,3-trihydroxypropane or the hydroxyl group of an ether thereof bears the acyl moiety:

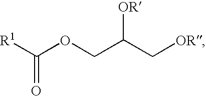

wherein R¹, R′, and R″ are as defined above.

As used herein, the term “β-monoglyceride” refers to an ester composed of a glycerol and an acyl group in which a hydroxyl group located at the 2-position of 1,2,3-trihydroxypropane or an ether thereof bears the acyl moiety:

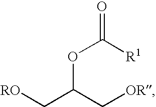

wherein R¹, R′, and R′ are as defined above.

As used herein, the term “1,2-diglyceride” refers to a diester composed of a glycerol as defined herein in which two adjacent hydroxyl groups at the 1- and 2-positions of 1,2,3-trihydroxypropane are esterified, or in which hydroxyl groups of adjacent ether groups at the 1- and 2-positions of 1,2,3-trihydroxypropane are esterified, or a combination thereof.

As used herein, the term “1,3-diglyceride” refers to a diester composed of a glycerol as defined herein in which two hydroxyl groups at the 1- and 3-positions of 1,2,3-trihydroxypropane are esterified, or in which hydroxyl groups of ether groups at the 1- and 3-positions of 1,2,3-trihydroxypropane are esterified, or a combination thereof.

As used herein, the term “capric/caprylic triglycerides” refers to a triester of 1,2,3-trihydroxypropane with a mixture of caprylic and capric acyl groups, which can also be referred to as medium chain triglyceride (MCT) oil. A “medium chain” refers to a fatty acyl group that contains between about 6 and about 14 carbon atoms. As is well known in the art, a hexanoyl group is also known as a caproic group, an octanoyl group is also known as a caprylic group, and a decanoyl group is also known as a capric group, and thus are all medium chain acyl groups.

As used herein, the term “hydrocarbon-based wax” refers to a type of lipid that may contain a wide variety of long-chain alkane esters, polyesters, and hydroxy esters of long-chain primary alcohols and fatty acids. They are usually distinguished from fats by the lack of triglyceride esters of glycerin (propan-1,2,3-triol) and three fatty acids.

As used herein, the term “hydrocarbon-based oil” refers to transparent, colorless, oils composed mainly of alkanes (typically 15 to 40 carbons) and cyclic paraffins. They are typically white, odorless, tasteless, waxy solids, with typical melting points between about 47° C. to 64° C. (116.6° F. to 147.2° F.).

Suitable exemplary hydrocarbon-based waxes and/or oils include squalane, dibutyl sebacate, light mineral oil, mineral oil, isopropyl laurate, isopropyl myristate, isopropyl palmitate, isopropyl strearate, octyl palmitate, myristyl alcohol, oleyl alcohol, oleic acid, myristyl lactate, diisopropyl adipate, octyldodecanol, caproic acid, caprylic acid, capric acid, glyceryl trioctante, C₁₂₋₁₅ alkyl benzoate, benzyl benzoate, tridecyl neopentanoate, castor oil, spermaceti, petrolatum, paraffin, and alpha terpineol.

As used herein, the term “petrolatum” refers to petroleum jelly; a yellow soft paraffin; a yellowish mixture of the softer members of the paraffin or methane series of hydrocarbons, obtained from petroleum as an intermediate product in the distillation; typically used as a soothing application to burns and abrasions of the skin, and as a base for ointments.

As used herein, the term “antioxidant” refers to a substance capable of slowing or preventing the oxidation of another substance. Suitable exemplary antioxidants include tocopherol, dl-alpha tocopherol, butylatedhydroxytoluene, butylatedhydroxyanisole, propyl gallate, tocopherol, tocopherol acetate, ascorbic acid, ascorbyl palmitate, and citric acid. Additional suitable exemplary antioxidants include butylated hydroxytoluene, ascorbic acid, sodium ascorbate, calcium ascorbate, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone and tocopherols such as vitamin E, rosemary, Irish moss extract, and limonene.

As used herein, the compound “dl-alpha tocopherol” refers to a compound having the following structural formula:

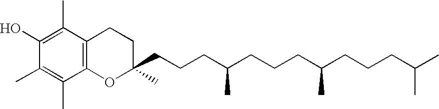

As used herein, the term “antimicrobial preservative” refers to a natural or synthetic chemical that is added to products such as foods, pharmaceuticals, paints, biological samples, wood, etc. to prevent decomposition by microbial growth. The antimicrobial preservative kills or inhibits the growth of microorganisms such as bacteria, fungi, or protozoans, as well as destroying viruses. Antimicrobial preservatives either kill microbes (microbicidal) or prevent the growth of microbes (microbistatic). Suitable exemplary antimicrobial preservatives include, e.g., potassium sorbate, sorbic acid, benzoic acid, potassium benzoate, methylparaben, propylparaben, butylparaben, benzyl alcohol, dimethylol-dimethyl hydantoin, imidazolidinyl urea, diazolidinyl urea, and methylisothiazolinone. Additional suitable exemplary antimicrobial preservatives include benzalkonium chloride, benzethonium, chlorhexidine, phenol, m-cresol, benzyl alcohol, methylparaben, propylparaben, chlorobutanol, o-cresol, p-cresol, chlorocresol, phenylmercuric nitrate, thimerosal, and benzoic acid.

As used herein, the term “diazolidinyl urea” refers to the compound chemically designated as N-[1,3-Bis(hydroxymethyl)-2,5-dioxo-4-imidazolidinyl]-N,N′-bis(hydroxymethyl)urea, having the following structural formula:

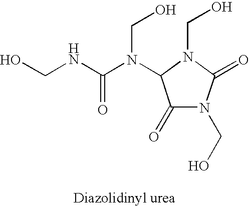

The composition described herein is considered to be “chemically stable” if it is not particularly reactive in the environment, not particularly reactive during normal use, and retains its useful properties on the timescale of its expected usefulness. In particular, the usefulness is retained in the presence of air, moisture or heat, and under the expected conditions of application. In this meaning, the composition is said to be unstable if it decomposes to a significant and appreciable degree, under the conditions of expected use or normal environmental conditions. Only stable compositions are contemplated herein.

As used herein, the term “topical” refers to body surfaces such as the skin.

As used herein, “lotion” refers to a liquid, usually an aqueous medicinal preparation containing one or more insoluble substances and applied externally for skin disorders.

As used herein, “cream” refers to an emulsified medicinal or cosmetic preparation; a semisolid emulsion of either the oil-in-water or the water-in-oil type, ordinarily intended for topical use.

As used herein, “gel” refers to a colloid in a more solid form than a solution; a jelly-like material formed by the coagulation of a colloidal liquid; many gels have a fibrous matrix and fluid filled interstices: gels are viscoelastic rather than simply viscous and can resist some mechanical stress without deformation.

As used herein, “ointment” refers to a salve or unguent for application to the skin, specifically a semisolid medicinal preparation usually having a base of fatty or greasy material; an ointment has an oil base whereas a cream is water-soluble.

As used herein, the term “topical skin patch” refers to an article of manufacture that includes a flexible backing having a front side and a back side and a formulation positioned on and/or in at least a portion of the front side of the backing. The formulation includes a therapeutically effective amount of the composition described herein. The formulation will also typically include an adhesive on and/or in at least a portion of the front side of the backing, sufficient to adhere the adhesive skin patch directly to a skin surface. The adhesive skin patch can either be occlusive or non-occlusive. In a specific embodiment, the adhesive skin patch can locally deliver the composition described herein, with minimal or no systemic delivery (and minimal or no systemic absorption by the mammal) of the composition.

As used herein, the term “essentially free of water” refers to less than about 10% w/w water, less than about 5% w/w water, less than about 1% w/w water, less than about 0.5% w/w water, or less than about 0.1% w/w water.

As used herein, the term “emulsifier” refers to a type of surfactant typically used to keep emulsion (mixtures of immiscible fluids) well dispersed. Emulsifiers typically have a hydrophobic (water-hating) tail and a hydrophilic (water-liking) head. The emulsifiers surround the immiscible molecule and form a protective layer so that the immiscible molecules cannot clump together. This action helps keeps the dispersed phase in small droplets and preserves the emulsion.

As used herein, the term “non-ionic emulsifier” refers to emulsifiers having no ionic groups.

Suitable exemplary non-ionic emulsifiers include cetearyl alcohol, ceteth-10, cetyl alcohol, and butylene glycol. A specific non-ionic emulsifier is emulsifying wax, mixtures of fatty acids of about 12 to 24 carbon atoms in length. Emulsifying waxes that are particularly suitable are those that meet the standards of the National Formulary (N.F.). A particularly suitable N.F. grade emulsifying wax is prepared from cetostearyl alcohol containing a polyoxyethylene derivative of a fatty ester of sorbitan. Emulsifying Wax N.F. is available from several manufactures, for example the emulsifying waxes sold under the trade names POLAWAXJ (Croda, Inc., NY) and LIPOWAXJ (Lipo Chemicals, Inc., Paterson, N.J.).

As used herein, the term “cationic emulsifier” refers to emulsifiers having a positive charge at the hydrophilic head of the emulsifier molecule.

Suitable exemplary cationic emulsifiers include fatty amines, quaternary ammonium compounds, as well as cationic copolymers, cationic mixed polymers, cationic polysaccharides, cationic cellulose derivatives, cationic or cationized hydrolyzed proteins such as collagen or keratin, and mixtures thereof. Specific examples of cationic emulsifiers include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, and the corresponding hydroxides thereof; quaternary esters, such as tetradecylbetaine ester chloride; diquaternary esters, such as dip almitoylethyldimethylammonium chloride; and diquaternary silicones.

As used herein, the term “anionic emulsifier” refers to emulsifiers having a negative charge at the hydrophilic head of the emulsifier molecule.

Suitable anionic emulsifiers include those based on sulfate, sulfonate, or carboxylate anions. Specific suitable exemplary anionic surfactant include sodium laureth sulfate, alkyl benzene sulfonates, soaps, fatty acid salts, and alkyl sulfate salts such as sodium lauryl sulfate, also known as sodium dodecyl sulfate, and ammonium lauryl sulfate.

As used herein, the term “amphoteric emulsifier” or “zwitterionic emulsifier” refers to emulsifiers having both positively charged and a negatively charged groups at the hydrophilic head of the emulsifier molecule.

Suitable exemplary amphoteric (zwitterionic) emulsifier include dodecyl betaine, dodecyl dimethylamine oxide, cocamidopropyl betaine, and cocoamphoglycinate.

As used herein, the term “buffer” refers to a mixture of a weak acid and its conjugate base or a weak base and its conjugate acid. Buffers have the property that the pH (acidity or basicity) changes very little when a small amount of acid or base is added to it. Buffers can be used as a means of keeping the pH at a nearly constant value in compositions described herein.

As used herein, the term “emollient” refers to substances that soften and soothe the skin. They are used to correct dryness and scaling of the skin. They differ from moisturizers in that moisturizers add moisture to the skin and emollient softens the skin. Both emollients and moisturizers are often present in topically applied products.

Suitable exemplary emollients include cetyl alcohol, isopropyl myristate and stearyl alcohol.

As used herein, the term “thickening agent” refers to substances which, when added to an aqueous mixture, increase its viscosity without substantially modifying its other properties. They provide body, increase stability, and improve suspension of added ingredients.

Suitable exemplary thickening agents include cellulose-based thickeners such as hydroxyethylcellulose, hydroxypropylcellulose or carbomer homopolymer thickeners including Carbopol® 934, 940, 941, 980, and 981 (Noveon, Inc., Akron, Ohio, USA).

As used herein, the term “anti-inflammatory agent” refers to a substance that reduces inflammation. Anti-inflammatory agents reduce pain by reducing inflammation. Anti-inflammatory agents can be based on steroids (steroidal) or non-steroidal.

As used herein, the term “moisturizer” refers to mixtures of chemical agents specially designed to make the external layers of the skin softer and more pliable, by increasing its water content by reducing evaporation. Moisturizers prevent and treat dry skin, protect sensitive skin, improve skin tone and texture, and mask imperfections.

As used herein, the term “retinoid” refers to a class of chemical compounds that are related chemically to vitamin A. The basic structure of a retinoid molecule consists of a cyclic end group, a polyene side chain and a polar end group. Examples of retinoids include retinal, tretinoin, isotretinoin, etreinate, and acitretin.

“Vitamin A” refers to a compound of the formula

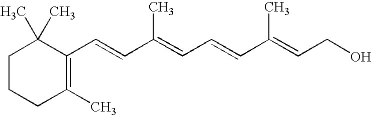

which is chemically designated as 3,7-dimethyl-9-(2,6,6-trimethyl-1-cyclohexen-1-yl)-2,4,6,8-nonatetraen-1-ol.

As used herein, the term “coal tar” refers to a viscous black liquid containing numerous organic compounds that is obtained by the destructive distillation of coal. Coal tar can be distilled into many fractions to yield a number of useful organic products, including benzene, toluene, xylene, naphthalene, anthracene, and phenanthrene. These substances, called the coal-tar crudes, form the starting point for the synthesis of numerous products—notably dyes, drugs, explosives, flavorings, perfumes, preservatives, synthetic resins, and paints and stains.

As used herein, the term “keratolytic agent” refers to a substance that causes desquamation (loosening) and debridement or sloughing of the surface cells of the epidermis.

“Salicylic acid” refers to 2-hydroxybenzoic acid (C₆H₄(OH)CO₂H), which is a colorless, crystalline organic carboxylic acid.

“Benzoyl peroxide” refers to a compound of the formula:

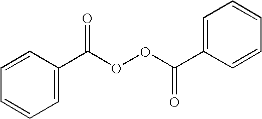

As used herein, the term “antifungal agent” or “fungicide” refers to a substance or chemical that will kill, destroy, inhibit, or inactivate a fungus to prevent growth. The chemical can be synthetic or biosynthetic and can include both organic and inorganic compounds. The fungicide can be a solid (e.g., powder), liquid, or a combination thereof. See, e.g., Concise Chemical and Technical Dictionary, Fourth Enlarged edition, Bennett, Chemical Publishing Company, NY, N.Y. (1986); and McGraw-Hill Concise Encyclopedia of Science & Technology, Fourth Edition, Parker, McGraw-Hill, NY, N.Y., (1998). Specifically, “fungicide” or “antifungal agent” can include a chemical that will kill, destroy, inhibit, or inactivate a eucaryotic microorganism to prevent growth. Exemplary eucaryotic microorganisms include algae, fungi, slime mold, protozoa, and eucaryotes in the microbial world.

As used herein, “fungi” or “fungus” refers to a large and diverse group of eucaryotic microorganisms whose cells contain a nucleus, vacuoles, and mitochondria. Fungi include algae, molds, yeasts, mushrooms, and slime molds. See, Biology of Microorganisms, T. Brock and M. Madigan, 6^{th} Ed., 1991, Prentice Hill (Englewood Cliffs, N.J.). Exemplary fungi include Ascomycetes (e.g., Neurospora, Saccharomyces, Morchella), Basidiomycetes (e.g., Amanita, Agaricus), Zygomycetes (e.g., Mucor, Rhizopus), Oomycetes (e.g., Allomyces), and Deuteromycetes (e.g., Penicillium, Aspergillus). One type of fungal infection is caused by a dermatophytic fungus. Examples of dermatophytic fungi are

Microsporum canis, Microsporum gypseum, Microsporum audouinii, Trichophyton tonsurans, Trichophyton mentagrophytes, Epidermophyton floccosum, and Trichophyton rubum. Other examples of fungal infections are those caused by Candida albicans or Candida guilliermondii.

As used herein, “algae” refers to a large and diverse assemblage of eucaryotic organisms that contain chlorophyll and carry out oxygenic photosynthesis. See, Biology of Microorganisms, T. Brock and M. Madigan, 6^{th} Ed., 1991, Prentice Hill (Englewood Cliffs, N.J.). Exemplary algae include Green Algae (e.g., Chlamydomonas), Euglenids (e.g., Euglena), Golden Brown Algae (e.g., Navicula), Brown Algae (e.g., Laminaria), Dinoflagellates (e.g., Gonyaulax), and Red Algae (e.g., polisiphonia).

As used herein, “mold” refers to a filamentous fungus, generally a circular colony that may be cottony, wooly, etc. or glabrous, but with filaments not organized into large fruiting bodies, such as mushrooms. See, e.g., Stedman's Medical Dictionary, 25^{th} Ed., Williams & Wilkins, 1990 (Baltimore, Md.). One exemplary mold is the Basidiomycetes called wood-rotting fungi. Two types of wood-rotting fungi are the white rot and the brown rot. An ecological activity of many fungi, especially members of the Basidiomycetes is the decomposition of wood, paper, cloth, and other products derived from natural sources. Basidiomycetes that attack these products are able to utilize cellulose or lignin as carbon and energy sources. Lignin is a complex polymer in which the building blocks are phenolic compounds. It is an important constituent of woody plants. The decomposition of lignin in nature occurs almost exclusively through the agency of these wood-rotting fungi. Brown rot attacks and decomposes the cellulose and the lignin is left unchanged. White rot attacks and decomposes both cellulose and lignin. See, Biology of Microorganisms, T. Brock and M. Madigan, 6^{th} Ed., 1991, Prentice Hill (Englewood Cliffs, N.J.).

As used herein, “yeast” refers to unicellular fungi, most of which are classified with the Ascomytes. See, Biology of Microorganisms, T. Brock and M. Madigan, 6^{th} Ed., 1991, Prentice Hill (Englewood Cliffs, N.J.).

As used herein, “mushrooms” refer to filamentous fungi that are typically from large structures called fruiting bodies, the edible part of the mushroom. See, Biology of Microorganisms, T. Brock and M. Madigan, 6^{th} Ed., 1991, Prentice Hill (Englewood Cliffs, N.J.).

As used herein, “slime molds” refers to nonphototrophic eucaryotic microorganisms that have some similarity to both fungi and protozoa. The slime molds can be divided into two groups, the cellular slime molds, whose vegetative forms are composed of single amoebalike cells, and the acellular slime molds, whose vegetive forms are naked masses of protoplasms of indefinite size and shape called plasmodia. Slime molds live primarily on decaying plant matter, such as wood, paper, and cloth. See, Biology of Microorganisms, T. Brock and M. Madigan, 6^{th} Ed., 1991, Prentice Hill (Englewood Cliffs, N.J.).

As used herein, the term “antibiotic agent” refers to a compound having activity against either Gram-positive or Gram-negative organisms (e.g., inhibits the growth or destroys the development of either Gram-positive or Gram-negative organisms). Stedman's Medical Dictionary, Illustrated, (25th Ed.), Williams & Wilkins: Baltimore (1990) and Mosby's Medical, Nursing, & Allied Health Dictionary, (5th Ed.), Mosby: St. Louis (1998).

Suitable antibiotic agents include, e.g., cilastatin, clavulanic acid, folinic acid, probenecid, pyridoxine, sulbactam, dapsone, ethambutol, isoniazid, pyrazinamide, rifampin, streptomycin, capreomycin, ethionamide, para aminosalicylic acid, cycloserine, ciprofloxacin, nalidixic acid, norfloxacin, ofloxacin, imipenam, meropenem, cilistatin, cefadroxil, cefazolin, cephalexin, cephalothin, cefaclor, cefamandole, cefonicid, cefoxitin, cefuroxine, cefoperazone, cefotaxime, ceftazidime, ceftizoxime, ceftriaxone, moxalactam, cefepine, bacitracin, vancomycin, aztreonam, amoxicillin, clavulanic acid, benzathine, penicillin g, penicillin v, ampicillin, carbenicillin indamyl, carbenicillin, mezlocillin, piperacillin, ticarcillin, cloxacillin, dicloxacillin, floxacillin, methicillin, nafcillin, oxacillin, colistmethate, polymixin b, trimethoprim, co-trimoxazole, mafenide, sulfadiazine, sodium sulfacetamide, sulfacytine, sulfadiazine, sulfamethoxazole , sulfapyridine, sulfasalazine, sulfisoxazole, chloramphenicol, clindamycin, spectinomycin, azithromycin, clarithromycin, erythrmoycin, erythromycin estolate, spiramycin, chlortetracycline, demeclocycline, doxycycline, minocycline, oxytetracycline, amikacin, kanamycin, neomycin, streptomycin, tobramycin, nitrofurantoin, griseofulvin, potassium iodide, fluconazole, itraconazole, ketoconazole, miconazole, clotrimazole, amphotericin b, nystatin, niclosamide, nifurtimox, piperazine, praziquantel, pyrantel pamoate, ascariasis, pinworm, thiabendazole, amodiaquine, chloroquine, hydroxychloroquine, mefloquine, primaquine, pyrimethamine, quinidine gluconate, fansidar, diloxanide furoate, melarsoprol, nifurtimox, paromomycin, pentamidine, sodium stibogluconate, suramin, metronidazole, foscarnet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, foscarnet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, and pharmaceutically acceptable salts thereof.

As used herein, the term “treating” or “treat” includes (i) preventing a pathologic condition from occurring (e.g. prophylaxis); (ii) inhibiting the pathologic condition or arresting its development; (iii) relieving the pathologic condition; and/or (iv) diminishing symptoms associated with the pathologic condition.

As used herein, the term “dermatologic condition” or “skin disorder” refers to disorders of the skin including, but not limited to, disease of the skin, skin condition, skin disease, skin problems, which include, but are not limited to, acne, eczema, psoriasis, rosacea, skin cancer, skin burns, skin allergies, congenital skin disorders, acantholysis, acanthosis, acanthosis nigricans, dermatosis, disease, erythroderma, furunculosis, impetigo, jungle rot, keratoderma, keratodermia, keratonosis, keratosis, keratosis nigricans, leukoderma, lichen, livedo, lupus, melanism, melanosis, molluscum, necrobiosis lipoidica, necrobiosis lipoidica diabeticorum, pemphigus, prurigo, rhagades, Saint Anthony's fire, seborrhea, vitiligo, xanthoma, xanthosis, Psoriatic arthritis, Reiter's syndrome, Guttate psoriasis, Dyshidriotic eczema, Acute and chronic graft versus host disease, Systemic sclerosis, Morphea, Spongiotic dermatitis, Allergic dermatitis, Nummular eczema, Pityriasis rosacea, Pityriasis rubra pilaris, Pemphigus erythematosus, Pemphigus vulgaris, Lichenoid keratosis, Lichenoid nitidus, Lichen planus, Lichenoid dermatitis, Seborrheic dermatitis, Autosensitization dermatitis, Dermatitis herpetiformis, and Eosinophilic dermatitis.

As used herein, the term “mammal” refers to a class of vertebrate animals of more than 15,000 species, including humans, distinguished by self-regulating body temperature, hair, and in the females, milk-producing mammae. Specifically, mammal may refer to a human, dog or cat. More specifically, mammal may refer to a human.

As used herein, the term “effective amount” or “therapeutically effective amount” is intended to include an amount of the composition described herein, to effectively treat or prevent the dermatologic condition, or to effectively treat the symptoms of the dermatologic condition, in a mammal.

As used herein, the term “psoriasis” refers to a chronic, non-contagious autoimmune disease which affects the skin and joints. It commonly causes red scaly patches to appear on the skin. The scaly patches caused by psoriasis, called psoriatic plaques, are areas of inflammation and excessive skin production. Skin rapidly accumulates at these sites and takes on a silvery-white appearance. Plaques frequently occur on the skin of the elbows and knees, but can affect any area including the scalp and genitals. In contrast to eczema, psoriasis is more likely to be found on the extensor aspect of the joint. “Plaque psoriasis” or “psoriasis vulgaris” is the most common form of psoriasis. It affects 80 to 90% of people with psoriasis. Plaque psoriasis typically appears as raised areas of inflamed skin covered with silvery white scaly skin. These areas are called plaques.

The term “hydrate” or “crystalline hydrate” refers to a substance (e.g., active pharmaceutical ingredient) that either contains no water (e.g., anhydrate), or contains molecular water, such that the hydration number is above 0. Suitable hydrates include, e.g., the anhydrate (hydration number, 0), hemihydrate (hydration number, 0.5), monohydrate (hydration number, 1), dihydrate (hydration number, 2), trihydrate (hydration number, 3), tetrahydrate (hydration number, 4), pentahydrate (hydration number, 5) and hexahydrate (hydration number, 6).

The term “polymorph” refers to a specific form or crystal structure of a compound. Polymorphs have different stabilities and may spontaneously convert from a metastable form (unstable form) to the stable form at a particular temperature. They also exhibit different melting points, solubilities (which affect the dissolution rate of drug and consequently its bioavailability in the body is also affected), X-ray crystal and diffraction patterns.

Any reference to any of the compounds described herein also includes a reference to a physiologically or pharmaceutically acceptable salt thereof. Examples of physiologically acceptable salts of the compounds described herein include salts derived from an appropriate base, such as an alkali metal (for example, sodium), an alkaline earth (for example, magnesium), ammonium and NX₄⁺ (wherein X is C₁-C₄ alkyl). Physiologically acceptable salts of a compound of an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound of a hydroxyl group include the anion of said compound in combination with a suitable cation such as Na⁺ and NX₄⁺ (wherein X is C₁-C₄ alkyl). For a list of exemplary suitable physiologically or pharmaceutically acceptable salts, see Pharmaceutical Salts, Stephen M. Berge, Lyle D. Bighley and Donald C. Monkhouse, Journal of Pharmaceutical Sciences, Vol. 66, No. 1, pp. 1-19 (1977) and Remington's Pharmaceutical Sciences, 17th ed., Mack Publishing Company, Easton, Pa., (1985), 1418.

### Solvent System

The present inventor has tested a number of the compositions disclosed in Didriksen and has determined that the vitamin D analog in these compositions is not relatively stable. As disclosed below in the Examples, stability was tested at accelerated conditions of 50° C. Under these conditions, degradation of the vitamin D analog was observed in compositions following one week of storage. Testing of an additional composition of Didriksen that was stored at 40° C. showed degradation of the vitamin D analog at 4 weeks.

In various embodiments, the present invention provides for a composition that includes a corticosteroid, a vitamin D analog, and a solvent comprising an acylated glycerol as the term is defined herein, that provides for a high degree of storage stability at high environmental temperatures.

The corticosteroid can be betamethasone dipropionate. The betamethasone dipropionate can be present in about 0.005% w/w to about 0.1% w/w of the composition. Specifically, the betamethasone dipropionate can be present in about 0.064% w/w of the composition. Additionally, 1.0 gram of the composition can include about 0.643 mg of betamethasone dipropionate, equivalent to about 0.5 mg of betamethasone.

The vitamin D analog can be calcipotriene. The calcipotriene can be present in about 0.0001% w/w to about 0.025% w/w of the composition. Specifically, the calcipotriene can be present in about 0.005% w/w of the composition.

The acylated glycerol can be a fatty acyl ester of a glycerol as the term is used herein, e.g., a mono-, di-, or tri-ester of 1,2,3-trihydroxypropane or a mono-, di-, or tri-ester of an ether thereof, or a combination thereof. Specifically, the acylated glycerol can be a mono-, di-, or tri-glyceride, that is, a mono-, di-, or triester of 1,2,3-trihydroxypropane. Alternatively, the acylated glycerol can be a mono-, di-, or tri-ester of a mono-, di-, or tri-ether of 1,2,3-trihydroxypropane, provided that at least one ester bond is present in the acylated glycerol molecule.

The acylated glycerol can be an α- or β-monoglyceride or, the acylated glycerol can be a 1,2- or a 1,3-diglyceride. Specifically, the acylated polyol can include one or more of a glycerol triester of at least one of caprylic and capric and linoleic acids, glycerol triacetate, or a glycerol monoester or diester of at least one of caprylic and capric acids. The acylated glycerol can be present in about 1% w/w to about 60% w/w of the composition.

The composition described herein can optionally further include a hydrocarbon-based wax or oil, such as petrolatum. The petrolatum can be present in about 90% w/w to about 99% w/w of the composition.

The composition described herein can optionally further include one or more of sorbitan sesquioleate, polysorbate 80, and propylene glycol. The sorbitan sesquioleate, polysorbate 80, and propylene glycol can be present in about 0.01% w/w to about 5% w/w of the composition.

The composition described herein can optionally further include an antioxidant, such as dl-alpha tocopherol. The dl-alpha tocopherol can be present in about 0.001% w/w to about 0.003% w/w of the composition.

The composition described herein can optionally further include an antimicrobial preservative, such as diazolidinyl urea. The diazolidinyl urea can be present in about 0.05% w/w to about 0.5% w/w of the composition.

The composition described herein is chemically stable. In a specific embodiment, the composition described herein is chemically stable when stored at about 40° C., for a period of about six weeks.

The composition described herein can be substantially non-aqueous. In a specific embodiment, the composition described herein can include less than about 5% w/w water.

The solvent system can include one or more specific solvents. For example, the solvent system can include one, two, three or four specific solvents. In one specific embodiment, the composition described herein can include one or more specific solvents, each independently having a relatively low dipole, a relatively large degree of hydrogen bonding, and being relatively oil miscible (e.g., not miscible in water). In such an embodiment, each of these specific solvents can amount to a single specific solvent (e.g., the single specific solvent has a relatively low dipole, has a relatively large degree of hydrogen bonding, and is relatively oil miscible). Alternatively, in such an embodiment, each of these specific solvents can amount to two specific solvents, three specific solvents, four specific solvents, etc.

In a specific embodiment, the solvent system can include a single specific solvent, such as an acylated glycerol, e.g., a capric/caprylic acid triglyceride. This single specific solvent has a relatively low dipole, has a relatively large degree of hydrogen bonding, and is relatively oil miscible. As such, the inclusion of additional specific solvents would not be necessary, due to the solvent system possessing a suitable low dipole, large degree of hydrogen bonding, and oil miscibility. However, in such an embodiment, the inclusion of additional specific solvents is contemplated within the composition described herein, as such additional specific solvents could enhance any one or more of the low dipole, large degree of hydrogen bonding, and oil miscibility of the solvent system.

The acylated glycerol employed as a specific solvent of the composition described herein can specifically includes acyl groups of any carbon-chain length, that can be branched or unbranched, or can be independently selected such that the acylated glycerol contains both branched and unbranched acyl groups. The acylated glycerol can be an acylated ether, such as an acylated hydroxyalkyl ether of 1,2,3-trihydroxypropane, for example an acylated pegylated 1,2,3-trihydroxypropane. In various embodiments, acyl groups substituting the etherified 1,2,3-trihydroxypropane moiety can be of any carbon-chain length, and can be branched or unbranched. In various embodiments, ether groups substituting the polyol can be of any carbon-chain length, and can be branched and unbranched.

The fatty acyl substitution of the glycerol can be a short chain (formyl, acetyl, propionyl), or medium chain, e.g., C₄-C₁₄, fatty acyl group that is fully saturated or is unsaturated, such as mono-, di-, or tri-unsaturated. Examples of suitable saturated fatty acyl groups include butyryl (C₄), caproic (C₆), caprylic (C₈), capric (C₁₀), lauric (C₁₂), and myristic (C₁₄). Alternatively, the fatty acyl group can be a long chain, e.g., C₁₄-C₃₀, fatty acyl that is fully or incompletely saturated.

Examples of suitable acylated glycerols to be used as the specific solvent of the composition described herein include a glycerol triester of at least one of caprylic and capric and linoleic acids (including products referred to by trade name “Miglyol 818”), glyceryl triacetate (including products referred to by trade name “Captex 500”), and the glycerol monoester or diester of at least one of caprylic and capric acids (including products referred to by trade name “Capmul MCM”). In various embodiments, sorbitan sesquioleate can also be present.

In another specific embodiment, the solvent system comprising an acylated glycerol can further include sorbitan sesquioleate. This solvent component has a relatively low dipole, has a relatively large degree of hydrogen bonding, and is relatively oil miscible. In various embodiments, the solvent system can include sorbitan sesquioleate present in about 0.01% w/w to about 5% w/w of the composition.

In another specific embodiment, the solvent system comprising an acylated glycerol can further include propylene glycol, which has a relatively low dipole and has a relatively large degree of hydrogen bonding. In various embodiments, the solvent system can further include propylene glycol present in about 0.01% w/w to about 5% w/w of the composition.

In another specific embodiment, the solvent system can further include polysorbate 80, which has a relatively low dipole and has a relatively large degree of hydrogen bonding. In various embodiments, the solvent system can further include polysorbate 80 present in about 0.01% w/w to about 5% w/w of the composition.

### Emulsifier

When the composition of the invention is an emulsion, the composition will preferably contain a primary emulsifier. Such emulsifier may be non-ionic, cationic, anionic, or zwitterionic.

Non-ionic emulsifiers include cetearyl alcohol, ceteth-10, cetyl alcohol, and butylene glycol. A preferred non-ionic emulsifier is emulsifying wax, mixtures of fatty acids of about 12 to 24 carbon atoms in length. Emulsifying waxes that are preferred are those that meet the standards of the National Formulary (N.F.). A preferred N.F. grade emulsifying wax is prepared from cetostearyl alcohol containing a polyoxyethylene derivative of a fatty ester of sorbitan. Emulsifying wax N.F. is available from several manufactures, for example the emulsifying waxes sold under the trade names POLAWAXJ (Croda, Inc., NY) and LIPOWAXJ (Lipo Chemicals, Inc., Paterson, N.J.).

Cationic emulsifiers include fatty amines; quaternary ammonium compounds; as well as cationic copolymers, cationic mixed polymers, cationic polysaccharides, cationic cellulose derivatives, cationic or cationized hydrolyzed proteins such as collagen or keratin, or a mixture thereof. Specific examples of cationic emulsifiers include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, and the corresponding hydroxides thereof; quaternary esters, such as tetradecylbetaine ester chloride; diquaternary esters, such as dip almitoylethyldimethylammonium chloride; and diquaternary silicones.

Anionic emulsifiers include but are not limited to those based on sulfate, sulfonate, or carboxylate anions. Examples of such anionic surfactants include sodium laureth sulfate, alkyl benzene sulfonates, soaps, fatty acid salts, and alkyl sulfate salts such as sodium lauryl sulfate, also known as sodium dodecyl sulfate, and ammonium lauryl sulfate.

Examples of amphoteric (zwitterionic) emulsifiers include but are not limited to dodecyl betaine, dodecyl dimethylamine oxide, cocamidopropyl betaine, and cocoamphoglycinate.

The amount of each of the corticosteroid and vitamin D analog, both in absolute terms as a percentage w/w of the composition and in relative terms (corticosteroid concentration/vitamin D analog concentration) will independently vary, depending on the particular corticosteroid and vitamin D analog included in the composition. In general, the concentration of the corticosteroid in the composition will be between about 0.001% and about 10.0% w/w of the composition, and the concentration of the vitamin D analog in the composition will be between about 0.001% and about 1.0% w/w of the composition. If desired, the concentrations of either or both of these components in the composition may be higher or lower than these ranges.

The composition can further optionally contain additional excipients used in topical pharmaceutical formulations, including but not limited to hydrocarbon-based waxes or oils such as squalane, dibutyl sebacate, light mineral oil, mineral oil, isopropyl laurate, isopropyl myristate, isopropyl palmitate, isopropyl strearate, octyl palmitate, myristyl alcohol, oleyl alcohol, oleic acid, myristyl lactate, diisopropyl adipate, octyldodecanol, caproic acid, caprylic acid, capric acid, glyceryl trioctante, C₁₂₋₁₅ alkyl benzoate, benzyl benzoate, tridecyl neopentanoate, castor oil, spermaceti, petrolatum, paraffin, and alpha terpineol; preservatives such as benzalkonium chloride, benzethonium, chlorhexidine, phenol, m-cresol, benzyl alcohol, methylparaben, propylparaben, chlorobutanol, o-cresol, p-cresol, chlorocresol, phenylmercuric nitrate, thimerosal, and benzoic acid; buffers; emollients such as cetyl alcohol, isopropyl myristate, stearyl alcohol; thickening agents such as a cellulose-based thickener like hydroxyethylcellulose or hydroxypropylcellulose or carbomer homopolymer thickeners including Carbopol® 934, 940, 941, 980, or 981 (Noveon, Inc., Akron, Ohio, USA); or anti-oxidants such as butylated hydroxytoluene, ascorbic acid, sodium ascorbate, calcium ascorbate, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone and tocopherols such as vitamin E, rosemary, Irish moss extract, and limonene.

The composition can further include additional chemical compounds that are useful in the treatment of skin disorders, such as psoriasis. For example, the composition may include one or more of an anti-inflammatory agents other than a corticosteroid. Additionally, the composition may further include a moisturizer, a retinoid (other than vitamin D), such as vitamin A. Additionally, the composition may further include coal tar, keratolytic compounds (such as salicylic acid and benzoyl peroxide), antifungal agents, and/or antibiotic agents.

The composition may be prepared in accordance with any of the numerous methods well known to the person skilled in the field of pharmacy. For example, a non-aqueous composition may be prepared by incorporating the components into an ointment or lotion base excipient, such as white soft paraffin. Specifically, preparation of a composition described herein may be performed by melting white soft paraffin, adding a solution of the vitamin D analog in the solvent system, followed by addition of a dispersion of the corticosteroid in an oil, such as paraffin oil, to obtain a mixture. Typical content ranges of the various components in the finished composition according to the invention are about 0.005 to about 0.1% w/w of the corticosteroid, from about 0.0001 to about 0.025% w/w of the vitamin D analog, and from about 1 to about 60% or higher w/w of the solvent system, the remainder typically being primarily base excipient, such as the above mentioned white soft paraffin and/or paraffin oil.

The composition may be used to treat dermatologic conditions that are responsive to topical treatment with either or both of a corticosteroid and vitamin D analog thereof. For example, the composition may be used to treat psoriasis.

In accordance with this method of medical treatment, the composition is topically applied to areas that are affected by a skin disorder, wherein the skin disorder is responsive to either or both of a corticosteroid and vitamin D analog. The composition is applied in an amount and for a period of time that is effective in treating the skin disorder, e.g., ameliorating the signs and/or symptoms of the skin disorder. Typically, such treatment involves application of the composition to affected sites one to three times daily for a period of time of at least two to three days and typically for longer durations such as for up to several months or longer.

All publications, patents, and patent applications are incorporated herein by reference. While in the foregoing specification this disclosed subject matter has been described in relation to certain preferred embodiments thereof, and many details have been set forth for purposes of illustration, it will be apparent to those skilled in the art that the disclosed subject matter is susceptible to additional embodiments and that certain of the details described herein may be varied considerably without departing from the basic principles of the disclosed subject matter.

### Examples

### Example 1

Summary: This example describes an alternate method of introducing both drug substances (API, Active Pharmaceutical Ingredient) into Calcipotriene 0.005% and Betamethasone Dipropionate 0.064% bulk Ointment. Whereas the original process adds betamethasone directly to the compounding kettle, but adds calcipotriene in solution as a side phase, the alternate method sequentially adds both APIs (calcipotriene and betamethasone dipropionate) to the same side phase, which is then added to the compounding kettle.

Exhibit Lot (Original) Process—In the original process, the two APIs are incorporated into the bulk separately and independently:

Betamethasone dipropionate is added as a dry powder directly into melted petrolatum contained in the compounding kettle. Mixing disperses the API.

Calcipotriene is first dissolved in a heated side-phase of caprylic/capric triglycerides, using high shear (rotor/stator) mixing to accelerate dissolution. The calcipotriene solution is then added to the compounding kettle in which betamethasone API has already been dispersed.

New (Refined) Process—In the refined process, both APIs are first added to the same side phase before transferring the side phase to the compounding kettle:

Calcipotriene is first dissolved in a heated side-phase of caprylic/capric triglycerides, using high shear, rotor/stator mixing to accelerate dissolution. Betamethasone dipropionate is then added as a dry powder to the same side phase in which calcipotriene has been dissolved. High shear, rotor/stator mixing disperses and suspends the betamethasone. The combined API side phase is then added to the compounding kettle containing melted petrolatum.

### Example 2

Calcipotriene/Betamethasone ointment is a combination of a vitamin D analog and a corticosteroid. These two active ingredients effectively treat skin plaques associated with psoriasis. Recently, formulations consisting of a triglyceride used to dissolve the calcipotriene have been formulated. The following composition was tested for stability of the two actives following storage of the product for four weeks at either 40° C. or 50° C. For betamethasone diproprionate 102% of the initial value was observed after four weeks at 40° C. while 101% of the initial value was observed after four weeks at 50° C. For calcipotriene 100% of the initial value was observed after four weeks at 40° C. and 98% of the initial value was observed after four weeks at 50° C.

Calcipotriene/Betamethasone Ointment
w/Capric/Caprylic Acid Triglyceride

| | % in | Wt (g) in |
|---|---|---|
| Formula | Formula | Formula |
| Calcipotriene | 0.0052 | 0.041 |
| Capric/Caprylic Acid | 2.25 | 18 |
| Triglyceride | | |
| Tocopherol | 0.002 | 0.02 |
| Active Phase Total | 2.51 | 20. |
| Petrolatum | 97.4 | 194.85 |
| Betamethasone | 0.064 | 0.128 |
| Diazolidinyl Urea | 0.1 | 0.2 |

### Example 3

For Examples 3-6: Calcipotriene/Betamethasone ointment is a combination of a vitamin D analog and a corticosteroid. These two active ingredients effectively treat skin plaques associated with psoriasis. Recently, formulations consisting of a triglyceride and a component used to dissolve the calcipotriene have been formulated. Various combinations have been used and in varying ratios ranging from a 90:10 ratio to a 50:50 ratio. Listed below are the formulations prepared.

The following composition was tested for stability of the two actives following storage of the product for six weeks at 40° C. For betamethasone diproprionate 94.2% of the initial value was observed after six weeks at 40° C. For calcipotriene 91.5% of the initial value was observed after six weeks at 40° C.

Calcipotriene/Betamethasone Ointment
w/Sorbitan Sesquioleate 90:10

| | % in | Wt (g) in |
|---|---|---|
| Formula | Formula | Formula |
| Calcipotriene | 0.0052 | 0.041 |
| Capric/Caprylic Acid | 2.25 | 18 |
| Triglyceride | | |
| Sorbitan Sesquioleate | 0.25 | 2 |
| Tocopherol | 0.002 | 0.02 |
| Active Phase Total | 2.51 | 20. |
| Petrolatum | 97.4 | 194.85 |
| Betamethasone | 0.064 | 0.128 |
| Diazolidinyl Urea | 0.1 | 0.2 |

### Example 4

The following composition was tested for stability of the two actives following storage of the product for six weeks at 40° C. For betamethasone diproprionate 91.1% of the initial value was observed after six weeks at 40° C. For calcipotriene 90.4% of the initial value was observed after six weeks at 40° C.

Calcipotriene/Betamethasone Ointment
w/Soribtan Sesquioleate 50:50

| | % in | Wt (g) in |
|---|---|---|
| Formula | Formula | Formula |
| Calcipotriene | 0.0052 | 0.042 |
| Capric/Caprylic Acid | 1.25 | 10 |
| Triglyceride | | |
| Sorbitan Sesquioleate | 1.25 | 10 |
| Tocopherol | 0.002 | 0.02 |
| Active Phase Total | 2.51 | 20. |
| Petrolatum | 97.4 | 194.56 |
| Betamethasone | 0.064 | 0.128 |
| Diazolidinyl Urea | 0.1 | 0.2 |

### Example 5

The following composition was tested for stability of the two actives following storage of the product for six weeks at 40° C. For betamethasone diproprionate 94.4% of the initial value was observed after six weeks at 40° C. For calcipotriene 92.1% of the initial value was observed after six weeks at 40° C.

Calcipotriene/Betamethasone Ointment w/Polysorbate 80 90:10

| | % in | Wt (g) in |
|---|---|---|
| Formula | Formula | Formula |
| Calcipotriene | 0.0052 | 0.042 |
| Capric/Caprylic Acid | 2.25 | 18 |
| Triglyceride | | |
| Polysorbate 80 | 0.25 | 2 |
| Tocopherol | 0.002 | 0.02 |
| Active Phase Total | 2.51 | 20. |
| Petrolatum | 97.4 | 194.67 |
| Betamethasone | 0.064 | 0.128 |
| Diazolidinyl Urea | 0.1 | 0.2 |

### Example 6

The following composition was tested for stability of the two actives following storage of the product for six weeks at 40° C. For betamethasone diproprionate 73.9% of the initial value was observed after six weeks at 40° C. For calcipotriene 54.7% of the initial value was observed after six weeks at 40° C.

Calcipotriene/Betamethasone Ointment w/Propylene Glycol 90:10

| | % in | Wt (g) in |
|---|---|---|
| Formula | Formula | Formula) |
| Calcipotriene | 0.0052 | 0.0412 |
| Capric/Caprylic Acid | | |
| Triglyceride | 2.25 | 18. |
| Propylene Glycol | 0.25 | 2 |
| Tocopherol | 0.002 | 0.02 |
| Active Phase Total | 2.51 | 20 |
| Petrolatum | 97.4 | 194.9 |
| Betamethasone | 0.064 | 0.128 |
| Diazolidinyl Urea | 0.1 | 0.2 |

### Example 7

Solvent Attributes

| | | | Miscibility in oil |
|---|---|---|---|
| Solvent | Dipolar | H-bonding | (mineral oil) |
| Capric/Caprylic Acid | Yes | Yes | Yes |
| Triglyceride | | | |
| Sorbitan Sesquioleate | Yes | Yes | Yes |
| Propylene Glycol | Yes | Yes | No |
| Polysorbate 80 | Yes | Yes | No |

While the invention has been described and exemplified in sufficient detail for those skilled in this art to make and use it, various alternatives, modifications, and improvements will be apparent to those skilled in the art without departing from the spirit and scope of the claims.

All patents and publications referred to herein are incorporated by reference to the same extent as if each individual publication was specifically and individually indicated to be incorporated by reference.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A composition comprising:
(a) a corticosteroid;
(b) a vitamin D analog; and
(c) an acylated glycerol.

2. The composition of claim 1, wherein the corticosteroid is betamethasone dipropionate, present in about 0.005% w/w to about 0.1% w/w of the composition.

3. The composition of claim 1, wherein the corticosteroid is betamethasone dipropionate, present in about 0.064% w/w of the composition.

4. The composition of claim 1, wherein the vitamin D analog is calcipotriene, present in about 0.0001% w/w to about 0.025% w/w of the composition.

5. The composition of claim 1, wherein the vitamin D analog is calcipotriene, present in about 0.005% w/w of the composition.

6. The composition of claim 1, wherein the acylated glycerol comprises an alkanoylated glycerol.

7. The composition of claim 1, wherein the acylated glycerol comprises an acylated hydroxyethyl ether of 1,2,3-trihydroxypropane or an acylated PEGylated 1,2,3-trihydroxypropane, or any combination thereof.

8. The composition of claim 1, wherein the acylated glycerol comprises a mono-, di-, or tri-glyceride.

9. The composition of claim 1, wherein the acylated glycerol comprises triesters of 1,2,3-trihydroxypropane and at least one of capric and caprylic acids.

10. The composition of claim 1, wherein the acylated glycerol comprises triesters of 1,2,3-trihydroxypropane and at least one of caprylic acid, capric acid, and linoleic acid.

11. The composition of claim 1, wherein the acylated glycerol comprises glycerol triacetate.

12. The composition of claim 1, further comprising petrolatum in about 90% w/w to about 99% w/w of the composition.

13. The composition of claim 1, further comprising sorbitan sesquioleate in about 0.01% w/w to about 5% w/w of the composition.

14. The composition of claim 1, further comprising polysorbate 80 in about 0.01% w/w to about 5% w/w of the composition.

15. The composition of claim 1, further comprising propylene glycol in about 0.01% w/w to about 5% w/w of the composition.

16. The composition of claim 1, further comprising dl-alpha tocopherol in about 0.001% w/w to about 0.003% w/w of the composition.

17. The composition of claim 1, further comprising diazolidinyl urea in about 0.05% w/w to about 0.5% w/w of the composition.

18. The composition of claim 1, that is chemically stable when stored at about 40° C., for a period of about four weeks.

19. The composition of claim 1, that is suitable for topical administration.

20. The composition of claim 1, wherein the composition is chemically stable when stored at about 40° C. for a period of at least about four weeks.

21. A method of treating a dermatologic condition in a mammal, the method comprising topically administering to a mammal in need of such treatment an effective amount of the composition of claim 1, to the affected topical area, for a period of time effective to treat the dermatologic condition.

22. A composition comprising:
(a) a corticosteroid;
(b) a vitamin D analog; and
(c) an acylated glycerol;
wherein the composition is chemically stable when stored at about 40° C. for a period of at least about four weeks.

23. A composition comprising:
(a) betamethasone dipropionate, present in about 0.005% w/w to about 0.1% w/w of the composition;
(b) calcipotriene, present in about 0.0001% w/w to about 0.025% w/w of the composition;
(c) triesters of 1,2,3-trihydroxypropane and at least one of capric and caprylic acids, wherein the triesters are present in about 1% w/w to about 60% w/w of the composition;
wherein the composition is chemically stable when stored at about 40° C. for a period of at least about four weeks.

24. The composition of claim 23, wherein:
(a) the betamethasone dipropionate is present in about 0.064% w/w of the composition; and,
(b) the calcipotriene is present in about 0.005% w/w of the composition.
